(19) United States
(12) Patent Application Publication
(10) Pub. No.: US 2009/0087848 A1
(43) Pub. Date: Apr. 2, 2009

(54) DETERMINING SEGMENTAL ANEUSOMY IN LARGE TARGET ARRAYS USING A COMPUTER SYSTEM

(75) Inventor: James Richard Piper, Aberlady (GB)

Correspondence Address: QUINE INTELLECTUAL PROPERTY LAW GROUP, P.C., P O BOX 458, ALAMEDA, CA, 94501, US

(73) Assignee: ABBOTT MOLECULAR, INC., Des Plaines, IL (US)

(21) Appl. No.: 12/180,406

(22) Filed: Jul. 25, 2008

Related U.S. Application Data
(63) Continuation-in-part of application No. 11/208,018, filed on Aug. 18, 2005.
(60) Provisional application No. 60/603,218, filed on Aug. 18, 2004.

(51) Int. Cl. C12Q 1/68; G06F 19/00; G01N 33/48; G01N 33/50

(52) U.S. Cl. 435/6; 702/19

(57) ABSTRACT

A method and/or system for making determinations regarding samples from biologic sources including statistical methods for making meaning grouping of observed data and/or for pre-selecting endpoints.

### CROSS REFERENCE TO RELATED APPLICATIONS

This application is a continuation-in-part of Ser. No. 11/208,018, filed Aug. 18, 2005, which claims priority from provisional patent application 60/603,218, filed 18 Aug. 2004 and incorporated herein by reference.

This application is related to U.S. patent application Ser. No. 10/269,723 filed 11 Oct. 2002, which is a non-provisional of 60/378,760 filed 12 Oct. 2001, both of which are incorporated herein by reference.

U.S. patent application Ser. No. 10/342,804 filed 14 Jan. 2003 and its corresponding provisional patent application 60/349,318, filed 15 Jan. 2002 are incorporated herein by reference.

### COPYRIGHT NOTICE

Pursuant to 37 C.F.R. 1.71(e), applicants note that a portion of this disclosure contains material that is subject to and for which is claimed copyright protection, such as, but not limited to, source code listings, screen shots, user interfaces, or user instructions, or any other aspects of this submission for which copyright protection is or may be available in any jurisdiction. The copyright owner has no objection to the facsimile reproduction by anyone of the patent document or patent disclosure, as it appears in the Patent and Trademark Office patent file or records. All other rights are reserved, and all other reproduction, distribution, creation of derivative works based on the contents, public display, and public performance of the application or any part thereof are prohibited by applicable copyright law.

### FIELD OF THE INVENTION

The present invention relates to the field of biologic assays and data analysis. More specifically, the invention relates to a computer or other logic processor implemented or assisted method for making certain determinations regarding assays, typically from biologic sources. In further embodiments, the invention involves systems, methods, or kits for performing screening and/or diagnostic tests for a variety of diseases or conditions.

### BACKGROUND OF THE INVENTION

Normal human cells contain 46 chromosomes in 22 autosome pairs and 2 sex chromosomes. Generally, normal cells contain two copies of every chromosome (other than the sex chromosome). Consequently normal cells also contain two copies of nearly every gene, except again for genes lying on the sex chromosomes.

In congenital conditions such as Down syndrome and in acquired genetic diseases such as cancer, this normal pattern of two copies of every chromosome and two copies of each gene is often disrupted. Whole chromosome number can be altered, with cancer cells in particular showing patterns of gain or loss of whole chromosomes or chromosome arms. (The number of copies of a chromosome in a cell is also referred to as its “ploidy”.) In other cases, a chromosomal rearrangement may result in a portion of one or more chromosomes being present in more than or fewer than two copies. This portion can correspond to whole or parts of one or more genes. Thus, genetic abnormalities are often described in terms of a gain or loss in copy number, where in different situations, copy number can refer to chromosomes, to genes, or more generally to contiguous sequences of DNA. Alterations in copy number may also be referred to as copy number imbalances. A contiguous sequence of DNA on one chromosome may be referred to as a segment.

Genes influence the biology of a cell via gene expression, which refers to the production of the messenger RNA and thence the protein encoded by the gene. Gene copy number is a static property of a cell established when the cell is created; gene expression is a dynamic property of the cell that may be influenced both by the cell's genome and by external environmental influences such as temperature or therapeutic drugs.

In general, various patterns of copy number imbalance are characteristic of certain congenital abnormalities or certain cancers, and determination of the pattern of imbalance can inform diagnosis, prognosis and/or treatment regimes. Thus, it is frequently desired to measure and/or determine and/or estimate copy number imbalance in cells and/or tissues and/or material derived therefrom. Chromosomal imbalances are measured using a variety of techniques, such as quantitative PCR, fluorescence in situ hybridization (FISH) measuring, and other techniques that attempt to count or estimate the number of specific genetic sequences. However, in many situations there is an increasing need for improved methods for detecting and/or measuring genetic imbalance.

While normal cells typically contain two copies of every non-sex linked gene, variations from this are found among many normal individuals. Improvements in array comparative genomic hybridization (array CGH) allow analyzing DNA copy number variations at a much higher resolution than previous techniques. With the increased resolution comes increased importance in identifying normal copy-number variation as compared to copy-number variation that may be associated with disease states. Measurement noise, however, has been one limitation restricting detection to polymorphisms that involve longer segments of the genome. Understanding copy number polymorphisms that are detectable by a particular array CGH technique is important so that normal variations are not falsely associated with disease.

In pre- and post-natal diagnoses, it is recognized that many defects in human development are due to gains and losses of DNA segments that occur prior to or shortly after fertilization. However, some gains and losses are increasingly recognized as being neutral in their effect and common in some families or genetically related groups.

Improved break-point detection is important for characterizing copy-number variations, as it has been found that normal or neutral variants can be inherited in families in generally a simple Mendelian fashion, and such variants at times may be characterized by a preservation of break-points in various individuals.

The discussion of any work, publications, sales, or activity anywhere in this submission, including in any documents submitted with this application, shall not be taken as an admission by the inventors that any such work constitutes prior art. The discussion of any activity, work, or publication herein is not an admission that such activity, work, or publication was known in any particular jurisdiction.

### REFERENCES

A. D. Carothers, A likelihood-based approach to the estimation of relative DNA copy number by comparative genomic hybridization, Biometrics 53, 848-856, 1997.
J. Clark et al, Genome-wide screening for complete genetic loss in prostate cancer by comparative hybridization onto cDNA microarrays, Oncogene 22, 1247-1252, 2003.
J. Fridlyand et al, Statistical issues in the analysis of the array CGH data, Proc. Computational Systems Bioinformatics CSB '03, 2003.
J. Fridlyand et al, Hidden Markov models approach to the analysis of array CGH data. J. Multivariate Analysis 90, 132-153, 2004.
I. Miller and M. Miller, John E. Freund's Mathematical Statistics 6^{th} edition. Prentice Hall, 1999.
J. Piper et al, An objective method for detecting copy-number change in CGH microarray experiments, Proc. 3^{rd} Euroconference on Quantitative Molecular Cytogenetics, Rosenön, Stockholm, Sweden, 4-6 Jul. 2002, pp. 109-114, 2002.
J. R. Pollack et al, Genome-wide analysis of DNA copy-number changes using cDNA microarrays. Nature Genet. 23, 41-46, 1999.

### SUMMARY

The present invention involves techniques, methods, and/or systems useful for analyzing data typically related to biologic samples and most typically implemented on some type of logic execution system or module. Various aspects of the present invention may be incorporated into software for running a number of analyses on biologic detection or diagnostic systems, such as microarray diagnostic systems. While a number of specific diagnostic assays and details thereof are described below, some of which have independently novel aspects, the analysis methods of the invention have application to a variety of diagnostic and/or predictive situations in which data sets must be analyzed to determine relevant groupings.

In specific embodiments, the invention is directed to research and/or clinical applications where it is desired to assay or analyze samples containing biologically derived material, such as cellular material or nucleic acids. The invention according to specific embodiments is further directed to applications where it is desired to analyze sample assays by analyzing images of assay reactions, for example, images of one of various types of array chips for biologic detection. In such a situation, the captured image data provides a digital representation of the observable data of the assay reaction. This image can be a two-dimensional image captured and analyzed within an information processing system, as will be understood in the art. According to embodiments of the invention, an image is digitally captured by and/or transmitted to an information processing system. FIG. 3 is an example of observed data captured as an array image with, for example, a reader either designed or modified for reading slides with different fluorescent labels.

Specific embodiments are directed to techniques, methods and/or systems that allow automatic segmental aneusomy detection (SA) (this is referred to as segmental aneuploidy detection is some earlier work and prior applications) in microarrays, in specific examples in Comparative Genomic Hybridization (CGH) microarrays and analysis of related data sets.

In further embodiments, the invention can be understood as method for detecting copy number change using an array and logic processing as described herein. Generally, the method involves capturing (which includes reading or receiving) a set of array image data. As would be well understood in the art, the array image data generally is embodied as ordered arrays of intensity values. For comparison, data is generally present from at least a test and a reference sequence. The array image generally includes a plurality of target locations, each corresponding to a particular biological target subsequence. While for clarity of explanation, terms commonly associated with array image data are used herein; any other data format for storing the relevant data can be employed. In an example method of the invention, ratio values of at least a test and reference sequence intensity value at target locations are determined. A non-modal segment is generally defined as a contiguous sequence of target locations with ratios different from an expected or normal value. Pre-scanning of the array image data is performed to determine candidate end-points (also referred to as start and end-points) for non-modal segments. According to specific embodiments of the invention, candidate end-points will comprise less than about 10% of the total number of array locations, thus making further analysis less computationally intensive. A ratio change is estimated that extends across a segment of adjacent targets, using one or more familiar techniques, such as a maximum likelihood analysis.

In more specific example analysis according to specific embodiments of the invention, the invention performs running window split averages along sequential array locations by calculating an average of a number of locations on either side of a selected location and detecting changes in average ratio between a first half and a second half of said running window split average. Changes that are significant are determined and indicated as candidate end-points for non-modal segments. This can be accomplished by performing an edge detection along sequential array locations and indicating locations at edges as candidate start and end-point locations for non-modal segments. Edge validity may be determined by a statistical technique that relates difference in average ratio between left and right halves of the window to variance of the ratio data. Maximum likelihood analysis is one analysis that may be used for detecting copy-number changes of segments.

With pre-scanning according to specific embodiments of the invention, the computation time for automatic segmental aneusomy detection (SA) is significantly reduced with larger number of targets. When SA treats segment start and end-points completely independently, the search for the best fit to a non-modal segment has computational costs that are quadratic in the number of targets on the chromosome The effect is that a 300-target SA analysis may take around a second on a standard personal computer, while an SA on a 300,000-target array would take something in the order of 10⁶ seconds (−2 weeks). Using the improved SA method of the invention and one or more further optimizations as discussed below, an the entire SA method becomes usable in practice, for example, requiring just one to a few seconds to compute to completion on a 667 Mhz PowerPC G4, even for a 300,000-target array.

The invention can also be embodied as a computer system and/or logic processing laboratory equipment to analyze CGH-array or similar data and this system can optionally be integrated with other components for capturing and/or preparing and/or displaying sample data.

Various embodiments of the present invention provide methods and/or systems for diagnostic analysis that can be implemented on a general purpose or special purpose information handling system using a suitable programming language such as Java, C++, Cobol, C, Pascal, Fortran, PL1, LISP, assembly, etc., and any suitable data or formatting specifications, such as HTML, XML, dHTML, SQL, TIFF, JPEG, tab-delimited text, binary, etc. In the interest of clarity, not all features of an actual implementation are described in this specification. It will be understood that in the development of any such actual implementation (as in any software development project), numerous implementation-specific decisions must be made to achieve a developer's specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems.

Furthermore, it is well known in the art that logic systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

When used herein, “the invention” should be understood to indicate one or more specific embodiments of the invention. Many variations according to the invention will be understood from the teachings herein to those of skill in the art.

### BRIEF DESCRIPTION OF THE DRAWINGS

FIG. 1A-E illustrate an example of building an iterative model from multiple chromosome hybridization data to identify segments of sequences of detected genetic imbalance according to specific embodiments of the invention.

FIG. 2 is an example graph comparing sensitivity versus specificity of imbalance detection using methods according to specific embodiments of the invention compared to other methods.

FIG. 3 is an example of observed data captured as an array image with, for example, a reader either designed or modified for reading slides with different fluorescent labels.

FIG. 4 is an example graph comparing sensitivity versus specificity for isolated-target segmental aneusomy (SA) by “slope” and “basic” methods according to specific embodiments of the invention.

FIG. 5 is a block diagram showing a representative example logic and/or diagnostic system in which various aspects of the present invention may be embodied.

FIG. 6 (Table 2) illustrates an example of diseases, conditions, or statuses for which substances of interest can be evaluated according to specific embodiments of the present invention.

### DESCRIPTION OF SPECIFIC EMBODIMENTS

### 1. Segmental Aneusomy Detection

Methods of the present invention can be most easily understood in the context of example diagnostic assays that are known in the art. Use of any specific examples of particular microarray system should not be taken to limit the invention, which has applications in related or analogous data collection and analysis situations.

In one known technique for detecting an imbalance in genes, chromosomes, or DNA segments of a test sample, the test sample is labeled with a first distinguishable label (e.g., a fluorophore such as Cy3) and a quantity of a reference sample of DNA is labeled with a second distinguishable label (e.g., Cy5). The two labeled samples are then hybridized together to a microarray that has been prepared with target sequence DNA areas or targets or spots arranged in a systematic way.

In one typical system, each spot of the microarray contains many copies of a known sequence of DNA, which are at times referred to as targets or target clones. In many systems, each target sequence will be represented by one or more replicate spots on the microarray. A test sample can be whole-genome DNA, for example derived from blood or amniotic fluid. A test sample can also be DNA or other bindable sequences from cancer tissue or other biological material. One earlier known human whole-genome microarray contains 3 replicate spots containing many clones of each of 333 target DNA sequences. Typically, each target DNA sequence contains a well-defined portion of a DNA sequence from a single chromosome.

More recently developed microarrays have substantially larger numbers of targets, each containing a smaller length sequence of DNA. One more recently developed human microarray contains replicate spots containing many clones for each of 30,000 target DNA sequences.

Thus, in a typical detection procedure using such a microarray, microarray target spots are hybridized with the test sample, reference sample and any other reagents (such as an excess of, for example, unlabeled competitor DNA (e.g., Cot1 DNA) to suppress hybridization signals from repeat sequence DNA). Images are captured, showing, for example, Cy3 and Cy5 fluorescence (corresponding to test sample and reference sample of DNA), at target spot areas. In this type of assay, the target spot intensities in the captured images represent the observable data from the assay. In example systems, captured images are typically corrected for artifacts such as background fluorescence, the spots segmented and identified, and the ratio of the test sample fluorescence to the reference sample fluorescence (e.g. Cy3 to Cy5) intensities is measured at each spot. Examples of such systems are described in the above referenced and incorporated patent applications. Following ratio normalization, the fluorescence ratios are expected to be about 1.0 for targets with corresponding (or genetically complementary) DNA sequences for which the copy number is the same in the test and reference samples, but different from 1.0 for spots for which the corresponding test DNA sequence copy number is in imbalance. An amplification or gain of copy number in the test sample will result in a larger ratio, while loss of copy number in the test sample will result in a lower ratio. In this discussion, the term ratio generally refers to normalized ratios. The ability to detect and/or localize smaller copy number changes is substantially affected by the number of targets and the DNA segment length of each target.

A variety of statistical methods have been proposed or employed to determine whether the ratio for a particular target sequence averaged across its replicates is significantly different from 1.0. One such is the “p-value” method, as described in the coassigned patent application referenced above (U.S. patent application Ser. No. 10/269,723, Piper, filed Oct. 11, 2002). That method, in some specific embodiments, computes three values: (1) a significance level or p-value from the average ratio of the replicates for one target; (2) the variance among the target's replicate spot ratios; and (3) the variance of the ratios of other targets on the same microarray that are assumed or known or predicted to have balanced DNA copy number (such targets can also be referred to as “modal” targets). The p-value method and some other statistical methods generally examine each target DNA sequence in isolation.

### Example Segmental Aneusomy (SA) Detection

In a first aspect, the present invention involves systems and/or methods that detect imbalanced regions of a genome using microarray data from target spots from one or more target DNA sequences. Particularly in the case of constitutional genetic imbalances such as those associated with congenital abnormalities, but also in many cancer samples, it is common for a DNA sequence copy number imbalance to affect a contiguous region of the genome sequence, for example the gain of a whole chromosome 21 in Down syndrome, or the deletion of several megabase pairs of DNA in a microdeletion syndrome. The invention in specific embodiments uses co-occurrence of imbalance in one or more contiguous targets to increase the sensitivity and specificity of imbalance detection.

In particular embodiments, the invention analyzes the set of observed spot ratios by iteratively determining models of expected ratios that best explain the observed ratios. An expected ratio is the ratio that would be observed for a target from a given copy number in the test sample and another given copy number in the reference sample in a perfectly noise-free system that has optimum sensitivity and no signal attenuation. Since the copy number of the reference DNA is known, the unknown copy number of the test DNA can be determined from the expected ratio. A model according to specific embodiments of the invention groups target sequences into sequential sets of target sequences on the same chromosome that all have the same expected ratio. Herein, these sequential sets are referred to as segments. The base model is that all target ratios have a ratio value of 1.0 (also referred to as modal targets).

In building a model according to specific embodiments of the invention, each iteration adds one non-modal segment of one or more target sequences to the previous model. The non-modal (or positive) segment that is chosen is the one that causes the new model to best fit the data, using an optimization based on the statistical concept of likelihood. The new model is accepted if and only if the gain in log-likelihood is statistically significant. When only non-significant changes to the model are possible, it is regarded as complete.

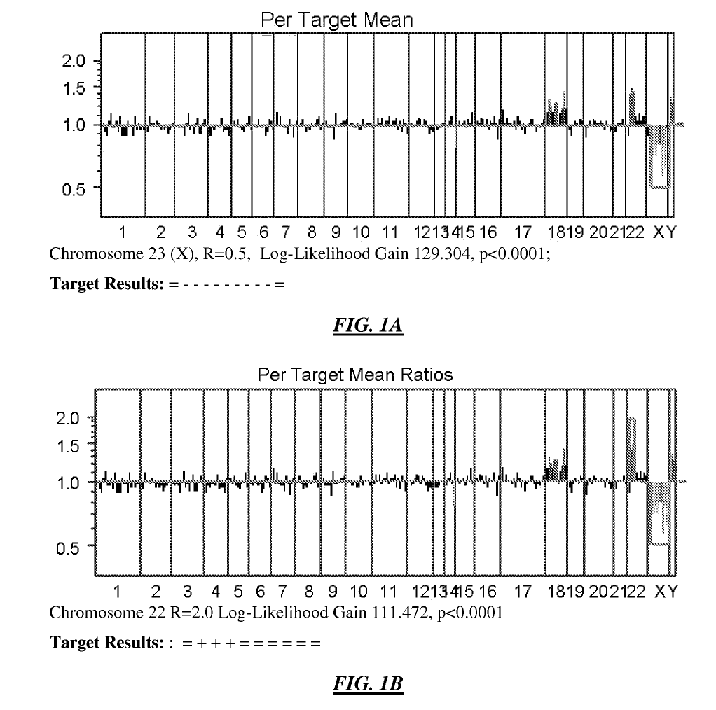

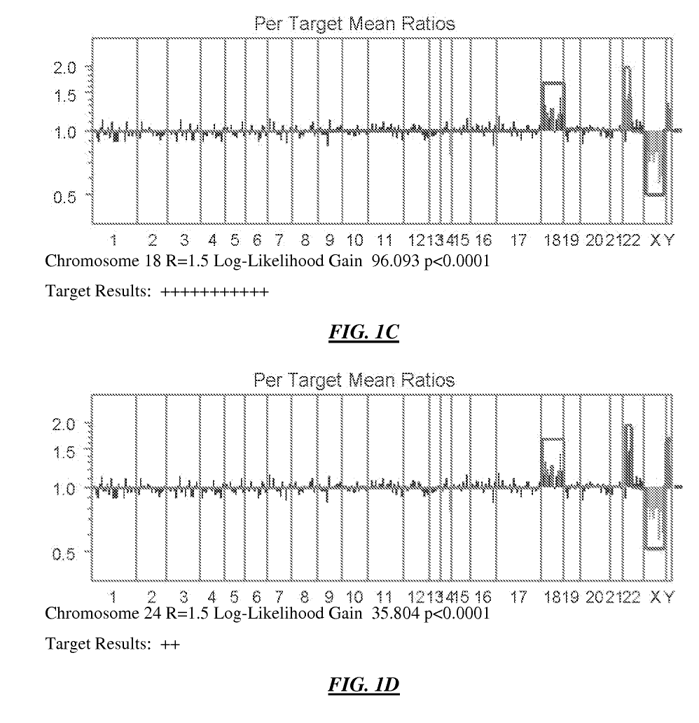

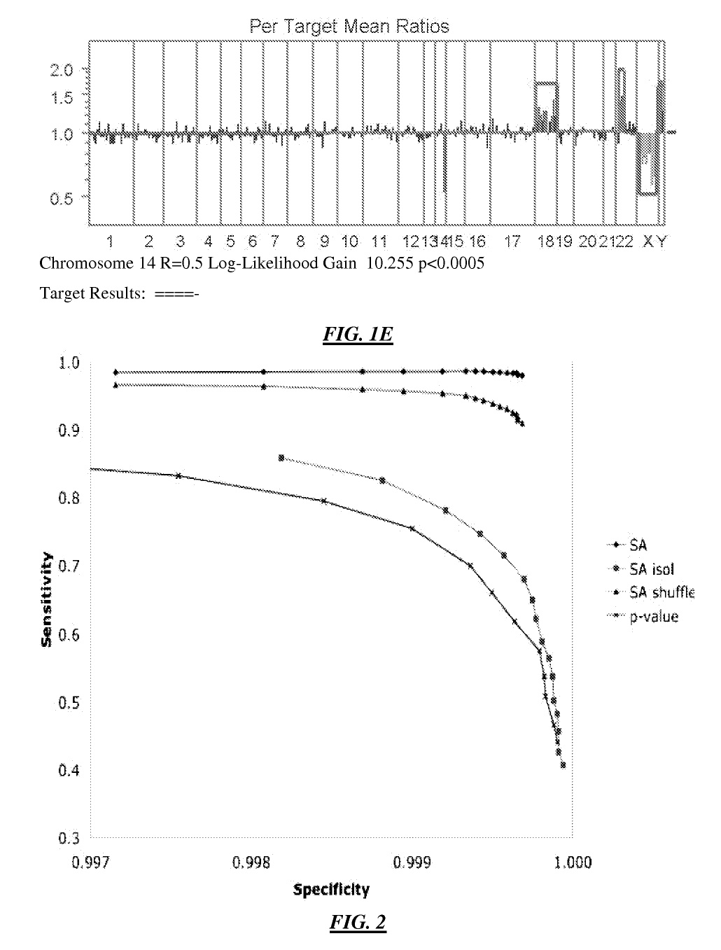

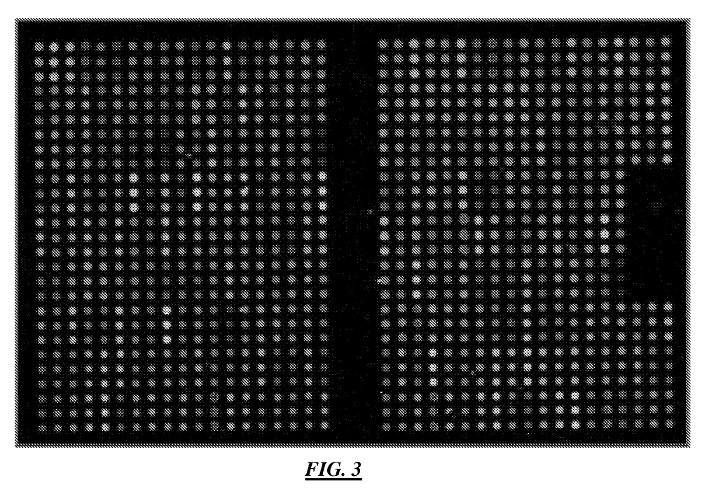

Model-building according to specific embodiments of the invention can be visually illustrated and conceptually understood by examination of FIG. 1A-E. FIG. 1A-E illustrate an example of building an iterative model from multiple chromosome hybridization data to identify segments of sequences of detected genetic imbalance according to specific embodiments of the invention. While the process is straightforward to illustrate, for some applications of this method, such as for validated and repeatable diagnostics, it is desirable to have a mathematically deterministic and rigorous method of performing the data analysis, examples of which according to specific embodiments of the invention are described further below.

In the sequence shown, each successive model fits the observed data significantly better than the preceding model. In this example, the gain in log-likelihood at the 6th iteration had p>0.02 by the χ² test familiar in the art of statistical analysis and was therefore judged not significant; this caused the search for better-fitting models to terminate.

Segmental aneusomy (SA) detection according to specific embodiments of the invention has better performance than the p-value method if positive targets (i.e., those targets for which the corresponding test sample sequence has a DNA loss or gain) form contiguous segments of length two target spots or more, and has at least equivalent performance in the detection of isolated positive target spots.

### Example Method

According to specific embodiments, the invention takes advantage of the fact that a test sample copy number change, whether involving a whole chromosome or part of a chromosome, usually will change the ratios at multiple sequential target spots. For purposes of this discussion, a contiguous set of DNA targets that all indicate the same copy number change in the test sample are referred to as a segmental change, or segment for short.

Methods of segment analysis have been considered in the context of applying cDNA clone expression microarrays to CGH analyses. The small sequence length of cDNA target clones results in very noisy ratio data when probed with whole-genome DNA, and the performance of individual targets is correspondingly poor. For example, Pollack et al (1999) described the use of “moving average windows” to detect single copy changes of sets of sequential cDNA target clones with 98% sensitivity and also 98% specificity, but did not apply any measure of significance to the detected segments. Clark et al (2003) proposed the use of Lowess curve fitting to the sequence of all target clone ratio data to detect possible segments with altered ratio, followed by the Mann-Whitney U test to provide a significance level for a candidate segment. One application of a segment technique to BAC/PAC clone microarrays specifically manufactured for CGH analysis was described by Fridlyand et al (2003, 2004), who fitted hidden Markov models (HMM) to the sequence of target ratios from array CGH analysis of cancer cell lines.

As Clark et al (2003) discussed, segment identification has two components. First, one or more candidate segments must be proposed. While an exhaustive search proposing all possible segments has been used in previous array analysis methods, this becomes increasingly impractical for CGH arrays having many thousands of targets and therefore many thousands of possible segment end-points. Thus, the first step in SA analysis according to specific embodiments of the invention is to determine a set of possible end-points of segments, or break-points, as described in more detail below, with pairs of identified end-points indicating candidate segments. Second, a measure of the value or significance of each candidate segment is used in order to choose good segments but reject less good segments, and thereby discriminate true copy number changes from the effects of random noise.

Finally, in specific embodiments, break-points identified with significant segments can be used to determine if a segment is likely a normal or benign copy number change.

Aspects of the present invention can be further understood with reference to a metaphase cell CGH analysis method described by Carothers (1997), who proposed a maximum-likelihood framework for iteratively building a model of a CGH chromosome ratio profile as a series of contiguous segments of profile points. In Carothers' model, every point in a given segment had the same test and reference copy numbers. Model construction was constrained to be consistent with the “crosstalk” between neighboring points on the chromosome profile, and employed a principle of parsimony, that the model was only allowed to become more complex if the resulting likelihood increase was significant according to an appropriate statistical test.

Specific embodiments of the present invention make use of one or more of: a likelihood framework, an iterative method, a parsimony principle, constraints, and the specification of the model in terms of underlying “expected ratios” derived from test and reference copy numbers. Crosstalk is generally not present on microarrays, and its role as a constraint on the solution has been replaced by (i) insistence that segments with non-modal expected ratios comprise sequential genomically-ordered target clones on the same chromosome, (ii) theory-based constraints on the allowable values of the expected ratios.

### 2. Pre-Selection of Candidate End-Points

Some array technologies (such as some Oligo CGH arrays) provide a very large numbers of target spots (for example, about 300,000 spots) of a smaller number of bases (about 20 or fewer bases), compared to, for example BAC arrays with 300 target spots of 100-150 kb. In such arrays, the ratio data is generally much noisier, but using the techniques of the invention this noise is compensated for by the much denser sampling of the genome.

However, to handle the large volume of data in such arrays, is challenging. If segment end-points are treated completely independently, a search for the best fit to a non-modal segment has a computational cost that is quadratic in the number of targets on the chromosome, which can require computation times of many hours in the case of dense arrays having hundreds of thousands of targets.

In specific embodiments, the invention involves a pre-pass that identifies a set of candidate end-points for non-modal segments. The search for complete segments is then limited by the candidate set. This search for end-points is essentially linear, not quadratic, in the number of targets. The search for complete segments is still quadratic in the number of candidate end-points, but since these form only a tiny fraction of the original set of loci on the array, the end result is execution in reasonable time. For example, a 300,000 target oligo CGH array was observed to generate about 3,000 end-points. Further optimizations of the candidate set can also be performed, as discussed below.

A candidate end-point search according to the invention produces a candidate set that is dramatically smaller than the entire set of loci, with a negligible false negative rate and a false positive rate that is as low as possible, though this is a secondary consideration, because false positive segment end-points will be comprehensively rejected by the original SA algorithm.

### Example Implementation

In a specific embodiment, a fixed-size window is applied (run) along the sequential set of target ratio data and at each location an average ratio between the first half of the window and the second half of the window is computed to look for a significant change. In one example, a simple mean between the first half and second half of, for example, a 20 locus window, can be used to identify a candidate segment end-point.

This type of analysis is analogous to edge detection familiar in signal or image processing techniques. The validity of the end-point can be measured by a suitable statistical technique that relates the difference in average ratio between the left and right halves of the window to the variance of the ratio data. The invention can use the same likelihood-based significance measure as is used by the overall SA analysis as described in incorporated references or a modification thereof (see below), or use a conventional t-test between the ratio distributions in the two window halves. With either of these techniques, a probability of an end-point can be assigned to each location. The set of candidate end-points is then obtained by applying a cut-off threshold to the probability of an end-point.

In specific example embodiments, the invention detects copy number changes by analyzing comparably large number of sequential DNA array locus ratios by pre-scanning ratios to determine a set of candidate end-point locations for non-modal segments, where such candidate locations comprise around 10% or less of the total number of array locations by estimating the ratio change that extends across a segment of adjacent targets; and using a maximum likelihood analysis in said estimation. Preselection in specific embodiments is performed using running window split averages along sequential array locations and detecting changes in average ratio between a first half and a second half of said running window split averages and statistically scoring or evaluating such changes to determine changes that are significant.

Alternatively, the invention can employ known or modified edge detection techniques and indicate locations at edges as candidate end-point locations for non-modal segments and can further measure validity of an edge by a statistical technique that relates difference in average ratio between left and right halves of the window to variance of the ratio data.

Algorithmically, in one example, the invention can be understood as pre-selecting by use of a conventional t-test, such as:
(a) retrieving ratio values in a window of length 2W, centered between a location i and a location i+1;
(b) calculating means mᵢ₁ and mᵢ₂ of the ratios (or log ratios) of locations in a first window half and in a second window half;
(c) calculating variances Sᵢ₁ and Sᵢ₂ of the ratios (or log ratios) of locations in said first window half and in said second window half;
(d) determining at value at location i as:

tᵢ=|mᵢ₁−mᵢ|/(sqrt((Sᵢ₁+Sᵢ₂)/W));
(e) determining Pᵢ, the significance of tᵢ in a 2-tailed t-test significance table with degrees of freedom equal to 2W−2;

The significance or probability value Pᵢ can be determined and stored for every location i for use later in the analysis. To determine a candidate set of locations i, a cut-off threshold can be applied.

In further embodiments, a likelihood analysis is used, for example comprising:
(f) retrieving ratio values in a window of length 2W, centered between a location i and a location i+1;
(g) calculating means mᵢ₁ and mᵢ₂ of the ratios (or log ratios) of locations in a first window half and in a second window half;
(h) calculating variances Sᵢ₁ and Sᵢ₂ of the ratios (or log ratios) of locations in said first window half and in said second window half;
(i) determining:
(j) Lᵢ=Σ_{j=i-W+1 i}((rⱼ−mᵢ₂)²/(Sᵢ₂+Sⱼ)−(rⱼ−mᵢ₁)²/(Sᵢ₁+Sⱼ))+Σ_{j=i+1 i+W}((rⱼ−mᵢₜ)²/(Sᵢ₁+Sⱼ)−(rⱼ−mᵢ₂)²/(Sᵢ₂+Sⱼ)), where the ratio (or log ratio) of the j'th target location is rⱼ with variance Sⱼ;
(k) wherein the first summation is a measure of the relative goodness of fit of all target ratios in the first half-window to segment ratio mil rather than to segment ratio mᵢ₂;
(l) wherein the second summation is a measure of relative goodness of fit of all target ratios in the second half-window to segment ratio mᵢ₂ rather than to segment ratio mil;
(m) determining a value of Lᵢ for every location i.

In some situations as described herein, the variance Sⱼ of the ratio of a single location j is unknown. In that instance, the invention in specific embodiments selects a plausible value and divides it by the number of original sample locations (or replicates of a sample location) that were averaged to produce the value for an averaged sample location. In truncated windows, for example close to a chromosome end or other end determined by the array, values are weighted to compensate for a shorter half-window.

In specific embodiments, if Lᵢ>T where T is a threshold, record both i and i+1 as potential segment end-points. Likewise, record the first and last targets on a chromosome or other array hard break as potential segment end-points.

Window sizes may be adjusted in different embodiments or in some cases in different passes or for different regions. For a 300,000 target oligo CGH array, appropriate sizes can include windows of approximately 10, 20, 40, 80, or other numbers selected in particular embodiments. One or more longer sizes may be selected to provide an analysis that is more immune to noise but may fail to detect short segments' end-points. One or more shorter sizes that give better detection of the short segment's end-points, better resolution of specific break-points, but at the expense of a higher risk of false positive signals resulting purely from noise.

In particular embodiments, underlying knowledge about particular DNA regions represented by target spots, such as regions subject to high benign variance, may be analyzed with different sized windows or different thresholds. Shorter windows may be used in regions where precise break-point location or precise copy change analysis is desired. Thus, a window size may be selected for an entire analysis or specific regions based on a particular problem being solved and/or data set being analyzed. For example, a larger window size may be selected for post-natal and some pre-natal analysis, where likely abnormalities have a small copy number change but are typically relatively extended in the genome. A larger window size may be selected for analysis of cancer samples, which have small segments having a large copy number and hence ratio change.

### Filtering Candidates

Further analysis may be done to reduce the number of candidate end-points. For example, sorting by order of edge strength per chromosome, and retaining only the top N per chromosome. Alternatively, where a number of adjacent high value locations are detected, retain local maximums.

### 3. Performing Maximum Likelihood Functions

One specific example of the likelihood function to be maximized can be understood as follows. (1) Let the genomically-ordered set of targets on the microarray be indexed by i, i=1 . . . k, and replicate spots within one target be indexed by r, r=1 . . . nᵢ. Typically nᵢ=a common integer (such as 3) for all i, and typically has values such as 333 or 287 or 30,000 or higher depending on the number of targets provided or analyzed on a particular microarray. Let the observed ratio data for a spot r belonging to target i be designated as yᵣᵢ, comprising an underlying value (constant across replicates for a target Yᵢ) plus an error term eᵢᵣ such that yᵣᵢ=Yᵢ+eᵢᵣ and the observed mean ratio across the replicate spots of target i is designated yᵢ and the set of observed ratios for the set of targets on the microarray is denoted y. (While log-ratios could be used, with only a slightly different theoretical development, in practice in tested situations, the log-ratio formulation did not perform as well as when using the ratios themselves.)

A model according to specific embodiments of the invention is a set of “expected ratios” denoted cᵢ representative of an underlying hypothesis about the test and reference copy numbers at each target locus. The set of expected ratios for the complete set of targets on the microarray is denoted c.

To choose the best fitting model by maximum likelihood, the invention maximizes the log-likelihood of y given c: L(c)=log(p(y|c))

Assume the target ratios are statistically independent of each other, specifically: p(yᵢ|c)=p(yᵢ|cᵢ) and p(yᵢ|cᵢ)=p(yᵢ|cᵢ,yⱼ), i≠j. This allows us to write: L(c)=log(p(y|c))=Σᵢp(yᵢ|cᵢ), the summation being taken across all targets i. Assuming normal distributions, L(c) can be computed from the formula: L(c)=a−Σᵢ(yᵢ−cᵢ)²/2vᵢ, where a is a constant, and vᵢ is the variance of yᵢ.

The variance vᵢ can be modeled as uᵢ+w, where uᵢ=within-target-variance/nᵢ (typically 3), and w is the “target noise” (variance among the set of targets of the target mean ratios when normal copy number test and reference DNAs are hybridized at all target loci).

Both E(var(yᵢ−yᵢ₋₁)) and E(uᵢ) can be estimated from the data. E(var(yᵢ−yᵢ₋₁)) is approximated by the variance of the set of all adjacent target ratio differences (yᵢ−yᵢ₋₁), denoted var{(yᵢ−yᵢ₋₁)}. When estimating var{(yᵢ−yᵢ₋₁)}, exclude the differences across segmental ratio changes, which of course are initially not known. This is achieved in specific embodiments by rejecting outlier differences, based on thresholds established from the first and third quartiles ±three times the interquartile range. Similarly, when computing the average within-target variance E(uᵢ), outlier variances are discarded.

Now maximize the likelihood L(c) over the set of possible values of c (expected target ratios), under constraints appropriate to the diagnostic analysis being performed.

In the present invention, the likelihood L(c) is computed only for segments whose end-points lie in the pre-selected set of end-points. This leads to a radical reduction in the computational cost of the segment analysis.

In an example embodiment, two constraints appropriate to particular CGH microarray diagnostic applications are used. First, all expected ratios cᵢ must either be 1.0, or must deviate from 1.0 by an amount that fits a model that the test and reference DNAs have copy numbers of 1, 2 or 3 everywhere. (While this constraint is particularly appropriate for congenital imbalances, other copy numbers may be more appropriate for detection of other cellular imbalances, such as those due to cancer, retroviral infection, or other conditions)

Note that the Y chromosome targets are not treated as having copy number zero in a female sample due to the high degree of homology between these targets and the X chromosome and/or autosome sequences. Instead, Y is assumed to have copy number of 0.5 in a female sample, leading to theoretically expected ratios of 0.5 in female test sample vs. male reference sample, 2.0 in male test sample vs. female reference sample, and 1.0 in sex-matched test and reference sample hybridizations. While this treatment of Y is a simplification, it has been found to work fairly well in practice, as has ignoring homologies other than between Y and X among targets.

In specific embodiments of the method, these constraints are applied by requiring that cᵢ=1+s(Rᵢ−1) where Rᵢ=tᵢ/rᵢ is one of {0.5, 1.0, 1.5, 2.0}, and s is a constant of the chip that will end up being estimated from the data. The s value in this discussion can be understood to represent the attenuation of a measured non-modal ratio as compared with the expected ratio value. This value is sometimes referred as a “slope” value as a result of some analogies to earlier work wherein measured ratio was plotted against expected ratio for a single experiment where there are different expected ratios, resulting in straight line with slope s. As a second constraint, while in principle, 0<s<1, to preclude trivial solutions, constrain s such that 0.25<s<1.0.

In further specific embodiments, the search proceeds by hypothesizing constrained changes to the expected ratios in the ordered sequence of targets. In each iteration, add whichever single non-modal segment (or new modal-ratio segment placed in the interior of an existing non-modal segment, e.g. in chromosome X) maximizes the likelihood L(c), by searching through a space defined by the following 4 free parameters:

1. t_{b}, the index of the first altered target.

2. tₑ, the index of the last altered target. The search is limited to segments contained within a single chromosome having pre-selected end-points.

3. q, the expected “ratio deviation” (i.e., from 1.0) of the altered targets assuming that slope=1. In specific embodiments, q is drawn from the set of 4 distinct allowed values expressed as (t/r−1), see above. Note that c=1+sq.

4. s, the current best estimate of slope for this chip.

The difference in the log-likelihood between the current and previous models, when multiplied by 2, is χ² distributed with degrees of freedom equal to the number of additional parameters added to the model (Miller and Miller, 1999, p. 404). Each iteration of model building is therefore evaluated by comparing twice the log-likelihood difference between current and previous models with the χ² distribution with 4 degrees of freedom. If the log-likelihood gain falls below the critical value for a chosen significance threshold, the search terminates. In other words, over-fitting of the model is avoided by use of a formal significance test.

In further specific embodiments, note that although the optimization may be done on a per-chromosome basis, slope s and target ratio variance w also have chip-wide components. Therefore, in specific embodiments, it is appropriate to search across the entire set of targets on the chip simultaneously, while not allowing potential segments to extend beyond the ends of the individual chromosome. The final result is a description of copy number changes for the entire chip.

The search space is relatively well constrained: t_{b} and tₑ must lie on the same chromosome and belong to the small set of pre-selected end-points; q can take only 4 possible values. As noted above, s is constrained to lie in the range 0.25<s<1.0. Brute-force search for optimal s with an increment in s of, say, 0.01 can be employed in specific embodiments. However, a preferred method is to note that L(c)=a−Σᵢ(yᵢ−cᵢ)²/vᵢ can be expressed as a function of s, as follows:

$$\begin{matrix}L ( c ) = a - {\sum }_{i} {( {y}_{i} - {c}_{i} )}^{2} / {v}_{i} \\ = a - {\sum }_{i} ( {y}_{i}^{2} - 2 {y}_{i} {c}_{i} + {c}_{i}^{2} ) / {v}_{i} \\ = a - {\sum }_{i} ( {y}_{i}^{2} - 2 {y}_{i} ( 1 + {sq}_{i} ) + {( 1 + {sq}_{i} )}^{2} ) / {v}_{i}\end{matrix} \qquad ( eqn 1 )$$

Given particular values of q, L_{b} and Lₑ at some given point in the search, the value of s which maximizes L(c) at those values can be found by differentiating the final expression above, and finding where the derivative is zero:

dL(c)/ds=−Σᵢ(−2yᵢqᵢ+2qᵢ+2sqᵢ²)/vᵢ, which is zero when

s=(Σᵢqᵢ(yᵢ−1)/vᵢ)/(Σᵢqᵢ²/vᵢ) (eqn 2)

If the optimum value of s lies outside the allowed range 0.25<s<1.0, then the triple {q, L_{b}, Lₑ} is eliminated from further consideration.

In further specific embodiments, equation 1 also provides a basis for efficient computation of L(c) in the subsequent iteration. Since at any one point in the search the current hypothetical next segment change is limited to a single chromosome, the value of L(c) contributed by each other chromosome is of the form Lⱼ(cⱼ)=Aⱼ+Bⱼs+Cⱼs², where j indexes the chromosome, cⱼ is the subset of c belonging to chromosome j, and Aⱼ, Bⱼ and Cⱼ are constants. The sums below are taken over all targets i belonging to chromosome j (symbolically, iεj):

Aⱼ=Σ_{iεj}(yᵢ−1)²/vᵢ

Bⱼ=−2Σ_{iεj}qᵢ(yᵢ−1)/vᵢ

Cⱼ=Σ_{iεj}qᵢ²/vᵢ

The terms Aⱼ are in any case constant throughout the analysis. While searching for a new segment in chromosome k, the invention can pre-compute the terms Σ_{j≠k}Bⱼ and Σ_{j≠k}Cⱼ, which immediately provide the contribution of the remaining 23 chromosomes to L(c) and its derivative with respect to s. With these optimizations, the entire SA method becomes usable in practice, for example, requiring just one or two seconds to compute to completion on a 667 Mhz PowerPC G4.

As an alternative to the method described above, instead of the value of slope s being re-estimated at each iteration of the algorithm as has been described, a segmental aneusomy detection algorithm can be implemented as follows: (1) Find the segment with the highest likelihood of being non-modal and compute the average of the observed ratios of the targets in the segment. Iterate this process until all segments whose likelihood gains are significant by the chi-square test have been found. (2) Find the best fit of the set of average observed segment ratios to the set of expected ratios. This step will estimate a value for the slope parameter s. The fitting must be constrained to plausible values of s. (3) Merge adjacent segments that have the same expected ratio. Segments detected at the first step that are allocated an expected ratio of 1.0 may indicate that the sample contains a mixed population of genomic clones (a “mosaic” sample). They should therefore not be discarded, and instead should be presented as anomalous to the user.

### 4. Experimental Results

In one set of experimental investigations, 515 microarray images were collected from experiments with microarrays containing either 287 targets or 333 targets, each with 3 replicate spots. The test DNAs used in these samples were mostly from various cell-lines which had either a known whole chromosome gain or a known microdeletion; a minority of samples used normal test DNA. 8 target clones previously identified as consistently (i.e., not randomly) and commonly being the cause of false positive or false negative detection events were excluded from the analysis of all samples using the microarrays that contained 287 targets; in the samples that used the microarrays with 333 targets, all target clones were included in the analysis.

Performance was evaluated in terms of the false negative rate (FNR) and false positive rate (FPR) on a target-by-target basis. FNR=FN/GTP, i.e., the number of false negative targets divided by the number of ground-truth positive targets. Missing targets were excluded from both numerator and denominator. Similarly FPR=FP/GTN. Results are mostly reported here in terms of analytical sensitivity (1−FNR) and analytical specificity (1−FPR).

In order to generate receiver operating characteristic (ROC; i.e., sensitivity vs. specificity) data, analyses were repeated with a wide range of χ² probability thresholds.

Because the available data sets consisted mostly of hybridizations by trisomy cell-lines, with relatively few examples of microdeletions, microduplications or other small imbalances, the target mean ratio data were analyzed in four different ways in order to simulate the issues that would be posed by small segments and isolated target copy number changes.

In one analysis, the SA method as described was applied to the set of target clone data in its original genomic order. This is referred to below as “standard SA”. In all microarrays with 287 targets, chromosome Y provided an example of a segment of length 2, and in a substantial number of samples the DiGeorge Syndrome deletion region of chromosome 22 was an example of a segment of length 3. All other non-modal segments had length 7 or more.

In a second analysis, the order of the target clones was permuted or “shuffled” into a reordering intended to separate at least some of the clones in long non-modal segments into segments of 1, 2, 3 or 4 adjacent clones. The permutation was semi-random so that a different reordering was used for each sample. The X and Y chromosomes were left unshuffled. The SA method as described was then applied to the set of target clone data in shuffled order. Sex chromosome targets were analyzed in the standard fashion, with segments allowed to be of any length, so that the slope estimation could “get off to a good start”. This is referred to below as “shuffled SA”.

In a third analysis, as a temporary measure for this simulation experiment only, the SA algorithm was additionally constrained so that the only possible candidate segments on autosomes consisted of single target clones. Thus every autosome target was potentially detectable as an isolated target only. This simulation provided a very large set of isolated targets, much larger than could be envisaged if real data had to be provided for this purpose. This is referred to as “isolated target SA”.

For comparison, the original p-value method (PV; for a full description, see Piper, 2002) was also applied, with FN counting restricted to the autosome ground truth positive targets only so that a direct comparison could be made with the isolated target method above.

In each case, FPR was based on all targets (i.e., including the sex chromosomes). FPR for isolated target SA was as generated by standard SA, because this generates more FPs than isolated target SA.

In order to get a clearer idea of the influence of segment length on performance, a two dimensional histogram of the number of target clones detected vs. the true length of a segment was extracted from the “shuffled SA” analysis. A single suitable value of the χ² probability threshold was used.

The constrained segmental aneusomy (SA) method described above is referred to as the “slope” method. There is a simpler alternative, which we refer to as the “basic” method. In the basic method, the ratio chosen to model any potential segment of observed ratio data is just the mean observed ratio across all the targets in the segment. In other words, this model has neither the notions of “allowed expected ratios” nor of “slope”. Preliminary experiments showed a high likelihood of false-positive segments containing just a few targets which randomly all had a small non-modal ratio “going in the same direction”, so a single ad hoc constraint proved to be necessary: that a segment's model ratio must be either <0.85 or >1.15.

### 5. Results and Discussion

FIG. 2 is an example graph comparing sensitivity versus specificity of imbalance detection using methods according to specific embodiments of the invention compared to other methods. FIG. 2 compares sensitivity versus specificity (also referred to as ROC) curves from the four methods: standard SA and shuffled SA on all targets, and isolated target SA and PV for autosome targets only. These results show clearly that SA performs better than PV; the improvement is dramatic if the copy number change involves segments of length two or more target clones. But the improvement is also substantial when SA is artificially limited to segments of length one target clone.

Table 1 illustrates the two-dimensional histogram of counts of non-modal segments present in the data analyzed by SA following target order “shuffling”, when the χ² threshold was chosen to give about one false positive per 3 microarrays. The histogram is indexed by a segment's true Length in the vertical direction, and by the number of target clones from the segment that were actually Detected in the horizontal direction. The results show that segment detection performance is excellent for segments with three or more target clones.

TABLE 1

| D | 0 | 1 | 2 | 3 | 4 | 5 | 6 | 7 | 8 | 9 | 10 | 11 | 12 | 13 | 14 |
|---|---|---|---|---|---|---|---|---|---|---|---|---|---|---|---|
| L1: | 586 | 1002 | | | | | | | | | | | | | |
| L2: | 156 | 25 | 1233 | | | | | | | | | | | | |
| L3: | 23 | 1 | 23 | 435 | | | | | | | | | | | |
| L4: | 1 | 2 | 7 | 16 | 175 | | | | | | | | | | |
| L5: | 0 | 0 | 0 | 2 | 6 | 99 | | | | | | | | | |
| L6: | 0 | 0 | 0 | 0 | 0 | 1 | 53 | | | | | | | | |
| L7: | 0 | 0 | 0 | 1 | 0 | 0 | 14 | 127 | | | | | | | |
| L8: | 1 | 0 | 0 | 0 | 0 | 0 | 0 | 1 | 74 | | | | | | |
| L9: | 1 | 0 | 0 | 1 | 0 | 1 | 0 | 1 | 29 | 414 | | | | | |
| L10: | 0 | 0 | 0 | 0 | 0 | 0 | 0 | 0 | 0 | 0 | 1 | | | | |
| L11: | 0 | 0 | 0 | 0 | 0 | 0 | 0 | 0 | 0 | 0 | 0 | 0 | | | |
| L12: | 0 | 0 | 0 | 0 | 0 | 0 | 0 | 0 | 0 | 0 | 0 | 0 | 0 | | |
| L13: | 0 | 0 | 0 | 0 | 0 | 0 | 0 | 0 | 0 | 0 | 1 | 0 | 7 | 20 | |
| L14: | 0 | 0 | 0 | 0 | 0 | 0 | 0 | 0 | 0 | 0 | 0 | 0 | 0 | 29 | 90 |

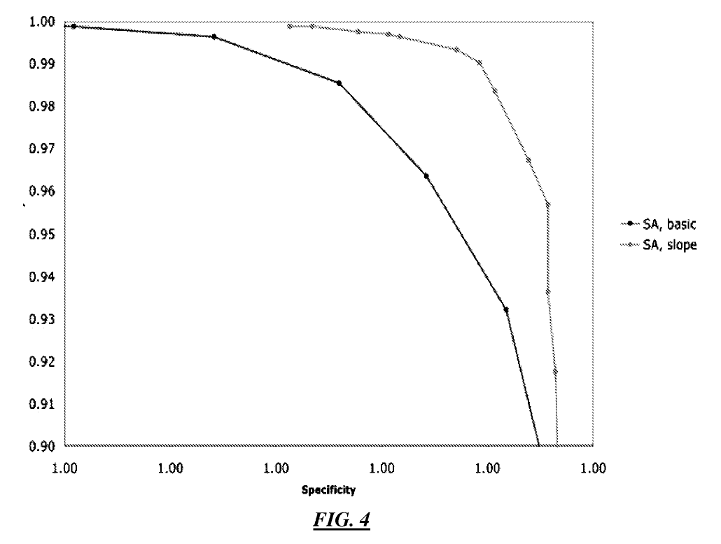

FIG. 4 is an example graph comparing sensitivity versus specificity for isolated-target segmental aneusomy (SA) by “slope” and “basic” methods according to specific embodiments of the invention. FIG. 4 shows ROC curves for isolated-target SA by the “slope” and “basic” methods, measured on a 110-chip subset of the data. The “slope” SA method outperforms the “basic” method in the detection of isolated target clones. This is believed to be chiefly due to the following. In order to be detected, a segment's log-ratio multiplied by the slope must be at least 50% of the smallest allowed model log-ratio. In other words, the method imposes a minimum ratio condition on the isolated clones. The minimum ratio is dependent on the slope and is therefore specific to each sample. Because of this, it eliminates false positives more efficiently than does the overall ratio threshold used by the “basic” method. The “basic” method does nevertheless have some advantages. Most notably, it will likely detect mosaic copy number changes rather better than the slope model.

### Example Application to Pre and Post-Natal Genetic Testing

In further embodiments, the invention can be used with array comparative genomic hybridization (aCGH) in clinical and/or research settings to detect segmental and whole chromosome changes in copy number. A particular specific example uses a Tecan HS4800 Hybridization Station in combination with the GenoSensor™ Reader. In one example embodiment, hybridizations are performed on an array containing 333 clones spotted in triplicate. In a preferred array, all telomeres and regions associated with known microdeletions/microduplications of interest are represented by two or more closely spaced target sequences on the array, with target specificity determined by analysis such as PCR or FISH against normal peripheral blood specimens (PBS) to avoid polymorphic targets. However, using candidate end-point identification, arrays containing 300,000 clone spots can be similarly analyzed.

According to specific embodiments of the invention, a user software package (e.g., the GenoSensor software) uses statistical analysis methods of segmental aneusomy (SA) as described herein to improve sensitivity and specificity. In further embodiments, an overall quality of hybridization indicator as described below can also be employed.

In experimental tests, this new array and assay format significantly reduces time to results detecting congenital genetic imbalances (e.g., pre-natal, post-natal, and pre-implantation) while improving assay performance.

Thus, in specific embodiments, a diagnostic system and/or method according to the invention can be optimized to detect chromosomal imbalances that are a common cause of developmental disorders such as mental retardation/developmental delay, physical birth defects and dysmorphic features. Currently, metaphase karyotype analysis is the gold standard in postnatal diagnostics of chromosome aneusomies, while fluorescence in situ hybridization (FISH) with probe(s) targeting submicroscopic genomic region(s) is the gold standard for detection of microdeletion and microduplication syndromes. The present invention in specific embodiments involves using comparative genomic hybridization (CGH) to in one assay diagnose chromosome aneusomies and microdeletion and microduplication syndromes. In specific embodiments, a detection system or method according the invention can be optimized for prenatal, postnatal, or embryonic pre-implantation diagnoses of these DNA sequence imbalances. Thus, in specific embodiments, the invention uses (Array-CGH) aCGH, (the application of CGH technology to chromosomal clones bound to a solid support) where each target clone is well-characterized and mapped to a specific chromosome region. An aCGH analysis according to specific embodiments of the invention allows highly sensitive detection of unbalanced genomic aberrations and can provide for the diagnostic detection of whole chromosome aneusomies, microdeletions, microduplications and unbalanced subtelomeric (subTel) rearrangements in a single assay.

The SA method of the invention can be used to enable a highly reproducible, automated aCGH assay format that does not require reciprocal hybridizations, and reliably detects copy number abnormalities (CNAs) from both fresh and fixed peripheral blood (PB) or cell line specimens.

### Automated Platform

In preferred embodiments, the analysis methods of the invention can be incorporated into a CGH platform that automates hybridization and washing, automates image capture and data analysis, assesses the quality of the assay, and reports qualitative results (gain, loss, no change). The following modifications can be used to enable some example current systems to perform according to the invention: a) modified microarray labeling/hybridization kit, b) extended-content microarrays on glass slides, c) Tecan HS4800 hybridization station running proprietary hybridization protocol, and d) GenoSensor slide reader with software algorithms including the methods described herein.

### Image and Data Analysis Software

In an example system, array images are captured with a reader modified for reading slides. Software associated with the reader controls image acquisition, analysis, and data reporting. The software identifies spots based on the DAPI signal, measures mean intensities from the green and red image planes, subtracts background, determines the ratio of green/red signal, and calculates the ratio most representative of the modal DNA copy number of the sample DNA. For each target, the normalized ratio, relative to the modal DNA copy number, is then calculated and the significance of the individual change reported.

Using segmental aneusomy analysis as described above allows for highly-sensitive detection of segmental CNAs. In addition, the software can include predictive quality control features, including a quantitative rating of overall assay and image quality (Quality Measure) as described below, and can also include such things as a measure of the completeness of spot segmentation and the reliability of spot identification, and image focus.

Thus, the new data analysis and quality rejection algorithms allow for a) rejection of poor quality data based on the experimentally selected cutoff for the Quality Measure parameter, and b) choosing the appropriate level of probability to count changes in genomic copy numbers as “real.”

### Other Diagnostic Uses

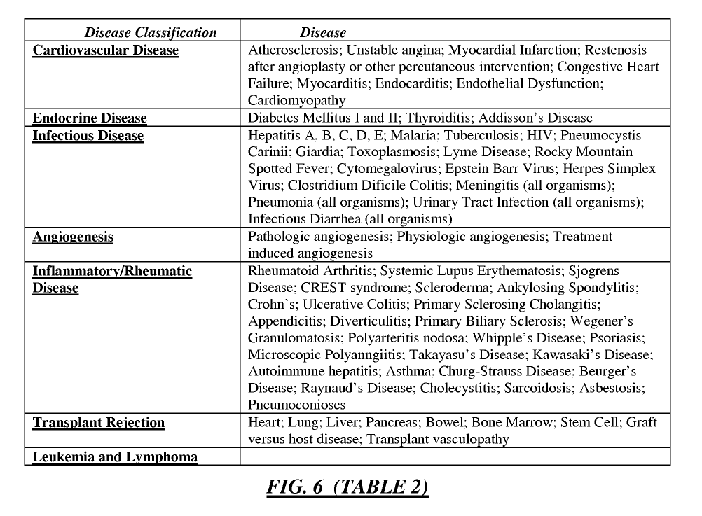

As described above, following identification and validation of a particular assay producing observable data sets, training statistical analysis parameters and selecting quality features as described above, assay analysis methods according to specific embodiments of the invention can be used in clinical or research settings, such as to predictively categorize subjects into disease-relevant classes, to monitor subjects for developmental disregulations, etc. Systems and/or methods of the invention can be utilized for a variety of purposes by researchers, physicians, healthcare workers, hospitals, laboratories, patients, companies and other institutions. For example, the invention can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine the most appropriate additional diagnostic testing for the patient, among other clinically and epidemiologically relevant applications. Essentially any disease, condition, or status for which an assay producing statistically analyzable data exists or can be developed can be more reliably detected using the diagnostic methods of the invention, see, e.g. Table 2. (FIG. 6).

In addition to assessing health status at an individual level, the methods and diagnostic sensors of the present invention are suitable for evaluating subjects at a “population level,” e.g., for epidemiological studies, or for population screening for a condition or disease.

### Web Site Embodiment

The methods of this invention can be implemented in a localized or distributed data environment. For example, in one embodiment featuring a localized computing environment, an assay reader according to specific embodiments of the present invention is configured in proximity to a desired diagnostic area, which is, in turn, linked to a computational device equipped with user input and output features. In a distributed environment, the methods can be implemented on a single computer, a computer with multiple processes or, alternatively, on multiple computers.

### Kits

A diagnostic assay according to specific embodiments of the present invention is optionally provided to a user as a kit. Typically, a kit of the invention contains one or more genetic targets constructed according to the methods described herein. Most often, the kit contains one or more DNA targets packaged or affixed in a suitable container. The kit optionally further comprises an instruction set or user manual detailing preferred methods of using the kit components for performing an assay of interest.

When used according to the instructions, the kit enables the user to identify diseases or conditions using patient tissues, including, but not limited to cellular interstitial fluids, whole blood, amniotic fluid, supernatant, etc. The kit can also allow the user to access a central database server that receives and provides information to the user and that may perform data analysis and or assay quality analysis. Additionally, or alternatively, the kit allows the user, e.g., a health care practitioner, clinical laboratory, or researcher, to determine the probability that an individual belongs to a clinically relevant class of subjects (diagnostic or otherwise).

### 6. Embodiment in a Programmed Information Appliance

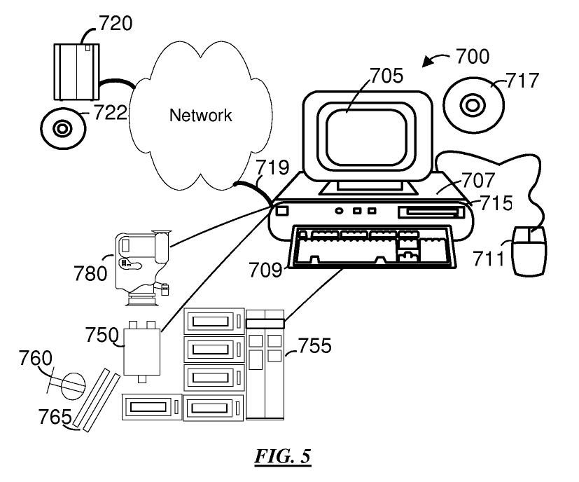

FIG. 5 is a block diagram showing a representative example logic device and/or diagnostic system in which various aspects of the present invention may be embodied. As will be understood from the teachings provided herein, the invention can be implemented in hardware and/or software. In some embodiments, different aspects of the invention can be implemented in either client-side logic or server-side logic. Moreover, the invention or components thereof may be embodied in a fixed media program component containing logic instructions and/or data that when loaded into an appropriately configured computing device cause that device to perform according to the invention. A fixed media containing logic instructions may be delivered to a viewer on a fixed media for physically loading into a viewer's computer or a fixed media containing logic instructions may reside on a remote server that a viewer accesses through a communication medium in order to download a program component.

FIG. 5 shows an information appliance or digital device 700 that may be understood as a logical apparatus that can perform logical operations regarding image display and/or analysis as described herein. Such a device can be embodied as a general purpose computer system or workstation running logical instructions to perform according to specific embodiments of the present invention. Such a device can also be custom and/or specialized laboratory or scientific hardware that integrates logic processing into a machine for performing various sample handling operations. In general, the logic processing components of a device according to specific embodiments of the present invention are able to read instructions from media 717 and/or network port 719, which can optionally be connected to server 720 having fixed media 722. Apparatus 700 can thereafter use those instructions to direct actions or perform analysis as understood in the art and described herein. One type of logical apparatus that may embody the invention is a computer system as illustrated in 700, containing CPU 707, optional input devices 709 and 711, storage media (such as disk drives) 715 and optional monitor 705. Fixed media 717, or fixed media 722 over port 719, may be used to program such a system and may represent a disk-type optical or magnetic media, magnetic tape, solid state dynamic or static memory, etc. The invention may also be embodied in whole or in part as software recorded on this fixed media. Communication port 719 may also be used to initially receive instructions that are used to program such a system and may represent any type of communication connection.

FIG. 5 shows additional components that can be part of a diagnostic system in some embodiments. These components include a viewer 750, automated slide or microarray stage 755, light (UV, white, or other) source 760 and optional filters 765, and a CCD camera or capture device 780 for capturing digital images for analysis as described herein. It will be understood to those of skill in the art that these additional components can be components of a single system that includes logic analysis and/or control. These devices also may be essentially stand-alone devices that are in digital communication with an information appliance such as 700 via a network, bus, wireless communication, etc., as will be understood in the art. It will be understood that components of such a system can have any convenient physical configuration and/or appearance and can all be combined into a single integrated system. Thus, the individual components shown in FIG. 5 represent just one example system.

The invention also may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

### Other Embodiments

The invention has now been described with reference to specific embodiments. Other embodiments will be apparent to those of skill in the art. In particular, a viewer digital information appliance has generally been illustrated as a personal computer. However, the digital computing device is meant to be any information appliance suitable for performing the logic methods of the invention, and could include such devices as a digitally enabled laboratory system or equipment, digitally enabled television, cell phone, personal digital assistant, etc. Modification within the spirit of the invention will be apparent to those skilled in the art. In addition, various different actions can be used to effect interactions with a system according to specific embodiments of the present invention. For example, an operator may speak a voice command, a key may be depressed by an operator, a button on a client-side scientific device may be depressed by an operator, or selection using any pointing device may be effected by the user.

It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this application, including any references filed as part of an Information Disclosure Statement, are incorporated by reference in their entirety.

What is claimed is:

1. A method to detect copy number change using a biological sequence array and a computer system comprising:
capturing a set of array image data, said array image data comprising an ordered array of intensity values from at least a test and a reference sequence, a target location of the array corresponding to a particular biological target subsequence;
acquiring ratio values of at least said test and said reference sequence intensity values at target locations of said array;
wherein a non-modal segment is a contiguous sequence of said target locations with ratios different from an expected or normal value;
pre-scanning said array image data to determine a set of candidate end-point locations for non-modal segments;
wherein said candidate end-point locations comprise less than 10% of the total number of array locations;
estimating the ratio change that extends across a segment of adjacent targets; and
using a maximum likelihood analysis in said estimation.

2. The method according to claim 1 further comprising:
performing running window split averages along sequential array locations, wherein said running window split averages comprise calculating an average of a number of locations on either side of a selected location; and
detecting changes in average ratio between a first half and a second half of said running window split averages;
determining changes that are significant changes;
indicating locations having significant changes as candidate end-point locations for non-modal segments.

3. The method according to claim 1 further comprising:
performing an edge detection along sequential array locations; and
indicating locations at edges as candidate end-point locations for non-modal segments.

4. The method according to claim 3 further comprising:
measuring validity of an edge by a statistical technique that relates difference in average ratio between left and right halves of the window to variance of the ratio data.

5. The method according to claim 4 further comprising:
measuring validity of an edge by using said maximum likelihood analysis as used for detecting copy-number changes of segments.

6. The method according to claim 2 further comprising:
retrieving ratio values in a window of length 2W, centered between a location i and a location i+1;
calculating means mᵢ₁ and mᵢ₂ of the ratios (or log ratios) of locations in a first window half and in a second window half;
calculating variances Sᵢ₁ and Sᵢ₂ of the ratios (or log ratios) of locations in said first window half and in said second window half;
determine:
Lᵢ=Σ_{j=i-W+1 i}((rⱼ−mᵢ₂)²/(Sᵢ₂+Sⱼ)−(rⱼ−mᵢ₁)²/(Sᵢ₁+Sⱼ))+Σ_{j=i+1 i+W}((rⱼ−mᵢₜ)²/(Sᵢ₁+Sⱼ)−(rⱼ−mᵢ₂)²/(Sᵢ₂+Sⱼ)),
where the ratio (or log ratio) of the j'th target location is rⱼ with variance Sⱼ;
wherein the first summation is a measure of the relative goodness of fit of all target ratios in the first half-window to segment ratio mil rather than to segment ratio mᵢ₂;
wherein the second summation is a measure of relative goodness of fit of all target ratios in the second half-window to segment ratio mᵢ₂ rather than to segment ratio mᵢ₁;
determine a value of Lᵢ for every location i.

7. The method according to claim 2 further comprising:
where variance Sⱼ of the ratio of a single location j is unknown,
select a plausible value and divide it by the number of original sample locations (or replicates of a sample location) that were averaged to produce the value for an averaged sample location.

8. The method according to claim 2 further comprising:
applying a standard statistical T-test to determine whether means of two samples (e.g., first and second half-windows) are significantly different.

9. The method according to claim 4 further comprising:
retrieving ratio values in a window of length 2W, centered between a location i and a location i+1;
calculating means mᵢ₁ and mᵢ₂ of the ratios (or log ratios) of locations in a first window half and in a second window half;
calculating variances Sᵢ₁ and Sᵢ₂ of the ratios (or log ratios) of locations in said first window half and in said second window half;
determining a Student's t value at location i as:
tᵢ=|mᵢ₁−mᵢ₂|/(sqrt((Sᵢ₁+Sᵢ₂)/W));
determining Pᵢ, the significance of tᵢ in a 2-tailed t-test significance table with degrees of freedom equal to 2W−2;
wherein said method comprises a conventional t-test between ratio distributions in the two window halves;
determining a value of Pᵢ for every location i.

10. The method according to claim 2 further comprising:
applying a cut-off threshold to edge strength to determine candidate locations.

11. The method according to claim 2 further comprising:
truncated windows close to a chromosome end;
weighting values in truncated windows to compensate for a shorter half-window.

12. The method according to claim 2 further comprising:
if Lᵢ>T where T is a threshold, record both i and i+1 as potential segment end-points.

13. The method according to claim 2 further comprising:
record the first and last targets on the chromosome as potential segment end-points.

14. The method according to claim 6 further wherein:
a window size is approximately 40;
threshold T is approximately 20.

15. The method according to claim 2 further comprising:
determining candidate points using a collection of different window sizes;
testing candidate points determined with different window sizes as non-modal segment end-points.

16. The method according to claim 15 wherein said different window sizes comprise two or more from the group:
approximately 10;
approximately 20;
approximately 40;
approximately 80.

17. The method according to claim 15 wherein said different window sizes are selected to comprise:
one or more longer sizes that is more immune to noise but fails to detect short segments' end-points.
one or more shorter sizes that give better detection of the short segment's end-points, but at the expense of a higher risk of false positive signals resulting purely from noise.

18. The method according to claim 6 further comprising:
selecting a window size corresponding to a particular problem being solved and/or data set being analyzed.

19. The method according to claim 6 further comprising:
selecting a larger window size for post-natal work, where likely abnormalities have a small copy number change but are typically relatively extended in the genome.

20. The method according to claim 6 further comprising:
selecting a smaller window size for analysis of cancer samples, which have small segments having a large copy number and hence ratio change.

21. The method according to claim 12 further comprising:
filtering candidate locations using one or more of:
sorting by order of edge strength per chromosome, and retaining only the top N per chromosome;
where a number of adjacent high value locations has been detected, retain local maximum.

22. The method according to claim 2 further comprising:
after determining significant non-modal segments;
fit the ratios of these segments to an expected ratio model and;
in the process extract the slope value.

23. The method according to claim 2 further comprising:
detecting mosaic changes in post-natal clinical applications by determining segments with a very small ratio change that fit the ratio ladder at the modal ratio point.
